(19) United States
(12) Patent Application Publication
(10) Pub. No.: US 2024/0102930 A1
(43) Pub. Date: Mar. 28, 2024

(54) Multiparametric Optical Method and Device for determining Uremic Solutes, including Uremix Toxins, in Biological Fluids

(71) Applicant: TALLINN UNIVERSITY OF TECHNOLOGY, Tallinn (EE)

(72) Inventors: Jurgen ARUND, Tallinn (EE); Risto TANNER, Tallinn (EE); Ivo FRIDOLIN, Tallinn (EE); Joosep PAATS, Tallinn (EE)

(73) Assignee: TALLINN UNIVERSITY OF TECHNOLOGY, Tallinn (EE)

(21) Appl. No.: 18/551,615

(22) PCT Filed: Mar. 31, 2022
(86) PCT No.: PCT/IB2022/053039

§ 371 Date: Sep. 21, 2023

(30) Foreign Application Priority Data
Mar. 31, 2021 (GB) 2104698.2

(51) Int. Cl. G01N 21/64

(52) U.S. Cl. CPC G01N 21/6428

(57) ABSTRACT

This invention relates to a novel method and a device for quantitative concentration measurements of protein bound and middle sized uremic toxins and advanced glycation end-products in the biological fluids, preferably in the spent dialysate. Invention combines unique spectral ranges of fluorescence and absorption to determine concentration of uremic toxins, such as indoxyl sulphate, beta-2-microglobulin and 4-pyridoxic acid in a way that provides significantly higher precision than previously known solutions.

### TECHNICAL FIELD

The invention relates to a method and a device for determining concentrations of uremic solutes, including uremic toxins, such as indoxyl sulphate, beta-2-microglobulin, 4-pyridoxic acid, in biological fluids, such as spent dialysate or urine, from the fluorescence and absorption of the light that has been directed to the biological fluid of interest.

### BACKGROUND ART

The concentration determination of certain uremic solutes and toxins in biological fluids with optical methods has been developed.

EP2585830B1 describes a device for determining concentration of middle molecule and protein bound uremic toxins, including beta-2-microglobulin, indoxyl sulphate, in biological fluids, such as spent dialysate, serum, urine and saliva. The device described in EP2585830B1 comprises of an optical module, comprising a fluorimetrical system, comprising a light source and a light detector, and a measuring fluorimetrical cuvette (cell) for holding a sample of the biological fluid so that the light can be directed onto the sample and the fluorescence signal can be detected from the sample; and a signal processing module consisting of a data acquisition module and a signal processing module incorporating concentration or removal calculation algorithms adapted to perform the transforming function, and a data representing module that is adapted for executing a program for data representation and comprises or is connected to a data visualization module. According to the invention the light source is operating in the wavelength range of 360-380 nm, and the fluorescence light detector is operating in the wavelength range of 440-470 nm, suitable for beta2-microglobulin measurements. Alternatively, the light source is operating in the wavelength range of 290-310 nm, and the fluorescence light detector in the wavelength range of 340-370 nm, suitable for indoxyl sulphate measurements. Whereby, the device may include flow-cuvette for receiving a flowing stream of the biological fluid.

EP2585830B1 describes a method for determining concentration of middle and protein bound uremic toxins in the biological fluids such as spent dialysate using the described device.

The disadvantage of the device and method is inadequate accuracy of determining concentration of uremic toxins, including indoxyl sulphate, beta-2-microglobulin, in the biological fluids, such as spent dialysate or urine, in comparison with the accuracy of clinical laboratory methods.

EP2746771B1 describes a device for measuring the concentration of a luminescent uremic substance in the spent dialysate. Whereby, the device comprises of a fluorimetrical system, incorporating a monochromatic light source and a light detector. In addition, the device comprises of an temperature sensor, optical filter of fluorescence or luminescence radiation, an optical beam path divider with a reference detector that are positioned after the light source, and an element for guiding the beam path for instance optical lens. Fluorescence or luminescence detector of the device is arranged at an angle to the original beam path of the light source.

The disadvantage of this known solution is the solution's indefinite description that does not include: the characterisation of the methodology for estimating concentration of uremic solute, accuracy and uncertainty of the solution, important working parameters that would allow to estimate the accuracy and uncertainty of the solution. signal processing module consisting of a data acquisition module and a signal processing module incorporating concentration or removal calculation algorithm adapted to perform.

EE05674B1 describes an apparatus and method for the quantitative determination of water soluble uremic solutes of low molecular weight, including urea, creatinine and uric acid in biological fluids, including spent dialysate. The device comprises of an optical module for determining the absorption spectrum of light, incorporating a measuring cuvette, a light source, a light detector; and a signal processing module. The signal processing module is adapted to execute a multiparametric concentration calculation algorithm. Whereby, the light source of the apparatus is operating in the wavelength range of 180-380 nm; and the device may include flow-cuvette.

The disadvantage of the device and method is that it is only capable to determine water soluble uremic solutes of low molecular weight using parameters of light absorption. The apparatus and method do not use fluorescence signal and are not capable to determine solutes that are protein bound uremic solutes, middle molecular weight uremic solutes, or advanced glycation end-products.

These three can be considered the closest known solutions from the state of the art in terms of methods and equipment appropriate to the invention.

Thus, there is a need for a new device and method which can determine concentration of uremic solutes, including indoxyl sulphate, beta-2-microglobulin, 4-pyridoxic acid, in biological fluids, such as in spent dialysate, urine, plasma, saliva, including in a flowing biological fluid, preferably on-line reagent-free determination, more accurately and reliably compared to the accuracy of the closest known solutions from the state of the art.

### BRIEF STATEMENT OF THE INVENTION

The objective of the invention is achieved with the method and device described below.

According to the invention, the method includes the following steps:
biological fluid, such as urine, spent dialysate, saliva or plasma, is directed or put into a measuring cell for holding a sample of the biological fluid;
containing at least two suitably selected wavelengths or wavelength ranges located in the most optimal region of the absorption and/or fluorescence spectrums of the respective uremic solutes (FIGS. 1 and 2; regions A and, F(I) and F(II), respectively), light is directed onto the biological fluid in the measuring cell;
the fluorescence signal is detected from the sample with a light detector at least from two suitably selected wavelengths or wavelength ranges;
signal intensities of the light absorption and/or fluorescence are determined from the detected light at suitably selected wavelengths or wavelength ranges;
the programme launched in the computational device uses a multiparameter algorithm for determining the content of uremic solutes, including toxins, in the biological fluid, utilising the light absorption and/or fluorescent signals and their inherent relationships as input, wherein
the first input of the algorithm A (FIG. 1) is the absorption of light in the wavelength range of λ=230-300 nm, which corresponds to the light absorbing substances found in biofluids (uric acid, hippuric acid), aromatic functional groups of amino acids in the composition of proteins (tryptophan, tyrosine, phenylalanine), and substances adsorbed on the surface of proteins (including hippuric acid, p-cresyl sulphate, indoxyl sulphate);
the second input of the algorithm F(I) (FIG. 2) is the fluorescence of substances in the wavelength range corresponding to the fluorescent uremic solutes found in biofluids (indoxyl sulphate, indole acetic acid, p-cresyl sulphate), fluorescent amino acids in the composition of proteins (tryptophan, tyrosine, phenylalanine), i.e. wavelength of the excitation light λEx=240-310 nm, wavelength of the emitted light λEm=270-420 nm; fluorescent substances adsorbed to the surface of proteins (including tryptophan, indoxyl sulphate, p-cresyl sulphate), i.e. wavelength of the excitation light λEx=240-310 nm, wavelength of the emitted light λEm=270-420 nm; and fluorescence of free uremic solutes in biological fluid (including tryptophan, indoxyl sulphate, p-cresyl sulphate), i.e. wavelength of the excitation light λEx=240-310 nm, wavelength of the emitted light λEm=270-420 nm.
the third input of the algorithm F(II) (FIG. 2) is the fluorescence of substances in the wavelength range corresponding to the advanced glycation end products, hereinafter AGE, including AGE pentosidine, AGE argpyrimidine, AGE vesperlysine, i.e. wavelength of the excitation light λEx=310-390 nm, wavelength of the emitted light λEm=360-600 nm;
and the fourth input of the algorithm is the weighting factors determined from previous empirically clinical trials.

The device may use flow-cuvette as a measuring cell.

For determining concentration of protein bound uremic toxins, including indoxyl sulphate, the following multiparametric algorithm is used:

C(IS)=a₀+α₁*f(A230 . . . 260)*F(Ex230 . . . 260Em360 . . . 420)+a₂*f(A260 . . . 300)*F(Ex260300Em390 . . . 460) (1)
according to which C(IS)—concentration of indoxyl sulphate; a0—absolute term of the function; a1, a2—the weighing factor of input parameters; A230 . . . 260, light absorption in a solution in the wavelength range of 230 nm to 260 nm; f(A230 . . . A260)—primary inner-filter effect correction function calculated in the region of absorption of the measured solution from 230 nm to 260 nm (Wang et al. 2017); F(Ex230 . . . 260Em360 . . . 420)—fluorescence of the measured solution in the wavelength range of excitation 230 nm to 260 nm and emission from 360 nm to 420 nm. A260 . . . 300, light absorption in a solution within wavelengths ranging from 260 nm to 300 nm; f(A260 . . . A300)—primary inner-filter effect correction function calculated in the area of absorption of the measured solution from 260 nm to 300 nm (Wang et al. 2017); F(Ex260 . . . 300Em390 . . . 460)—fluorescence of the measured solution in the range of wavelengths: excitation from 260 nm to 300 nm and emission from 390 nm to 460 nm; coefficients a0, a1 and a2 are coefficients that are empirically determined from clinical trials, i.e. parameters that are dependent on the optical design of the system.

For determining concentration of uremic solutes associated with AGE products, including 4-pyridoxic acid (4PA), the following multiparametric algorithm is used:

C(4PA)=c₀+c₁*F(Ex310330Em360 . . . 420)+c₂*F(Ex310 . . . 330Em420 . . . 600) (2)
according to which C(4PA)—concentration of 4-pyridoxic acid; c0—absolute term of the function; c1, c2—the weighing factor of input parameters; F(Ex310 . . . 330Em360 . . . 420)—fluorescence of the measured solution in the wavelengths range of excitation 310 nm to 330 nm and emission from 360 nm to 420 nm; F(Ex310 . . . 330Em420 . . . 600)—fluorescence of the measured solution in the wavelengths range of excitation 310 nm to 330 nm and emission from 420 nm to 600 nm; coefficients c0, c1 and c2 are coefficients that are empirically determined from clinical trials, i.e. parameters that are dependent on the optical design of the system.

For determining concentration of middle molecular weight uremic toxins, including beta-2-microglobulin, the following multiparametric algorithm is used:

C(b2M)=b₀+b₁*A260 . . . 290+b₂*f(A260 . . . 290)*F(Ex260 . . . 290Em290 . . . 360)+ . . . b₃*F(Ex320 . . . 380Em470 . . . 600) (3)
according to which: C(b2M)—concentration of beta-2-microglobulin; b0—absolute term of the function; b1, b2, b3—the weighing factor of input parameters; A260 . . . 290—light absorption in a solution in the wavelength range of 260 nm to 290 nm; f(A260 . . . 290)—primary inner-filter effect correction function calculated in the region of absorption of the measured solution from 260 nm to 290 nm (Wang et al. 2017); (Wang et al. 2017); F(Ex260 . . . 290Em290 . . . 360)—fluorescence of the measured solution in the wavelength range of excitation 260 nm to 290 nm and emission from 290 nm to 360 nm. F(Ex320 . . . 380Em470 . . . 600)—fluorescence of the measured solution in the wavelengths range of excitation 320 nm to 380 nm and emission from 470 nm to 600 nm; coefficients b0, b1, b2 and b3 are coefficients that are empirically determined from clinical trials, i.e. parameters that are dependent on the optical design of the system.

The objective of the invention is achieved by a device that comprises: at least one measuring cell—a measuring cuvette that passes through each measuring cell for storing the biological fluid to be measured, each measuring cell contains a light source for directing light to the biological fluid, the first light detector for detecting light absorbed in the biological fluid, and the second light detector for detecting light emitted from the biological fluid due to fluoresecne, and a signal processing module containing a data collection module and a computational device for processing the collected data, whereby the device is set up to use the method described above.

### BRIEF DESCRIPTION OF FIGURES

FIG. 1 is a graph depicting the average UV absorption spectrum (absorption spectrum) of spent dialysate samples (N=65) collected 7 minutes after the start of hemodiafiltration treatment.

FIG. 2 is a graph depicting the average emission spectra of spent dialysate samples (N=65) collected 7 minutes after the start of hemodiafiltration treatment at the excitation wavelengths Ex 240 nm, Ex 280 nm and Ex 320 nm.

FIG. 3 is a diagram shows one of the possible embodiments of the device according to the present invention from the top.

FIG. 4 is a diagram that shows the device that depicted in FIG. 3 in side view.

FIG. 5 is a block diagram that shows of one of the embodiment examples of the invention.

FIG. 6 is a graph that shows values of coefficient of determination found between concentration of indoxyl sulphate, 4-pyridoxoxic acid and beta-2 microglobulin in spent dialysate samples (N=369), collected 7, 60, 120, 180, 240 min after the start of hemodiafiltration treatment and from the tank, and the corresponding light absorption values in the region of 200-400 nm with the increment of 1 nm.

FIG. 7 is a graph that shows the determination coefficients found between the values of the fluorescence signals of spent dialysate samples (N=369), collected 7, 60, 120, 180, 240 min after the start of hemodiafiltration treatment and from the tank, and the concentration of protein-bound indoxyl sulphate at the excitation wavelength between 200 and 400 nm with the increment of 10 nm and emission wavelength between 250 and 600 nm with the increment of 5 nm.

FIG. 8 is a graph that shows the determination coefficients found between the values of the fluorescence signals of spent dialysate samples (N=369), collected 7, 60, 120, 180, 240 min after the start of hemodiafiltration treatment and from the tank, and the concentration of beta-2-microglobulin at the excitation wavelength between 200 and 400 nm with the increment of 10 nm and emission wavelength between 250 and 600 nm with the increment of 5 nm.

FIG. 9 is a graph that shows the determination coefficients found between the values of the fluorescence signals of spent dialysate samples (N=369), collected 7, 60, 120, 180, 240 min after the start of hemodiafiltration treatment and from the tank, and the concentration of 4-pyridoxic acid at the excitation wavelength between 200 and 400 nm with the increment of 10 nm and emission wavelength between 250 and 600 nm with the increment of 5 nm.

FIGS. 10A and 10B are graphs that depict a comparison of indoxyl sulphate's assessment methodologies on a calibration set; where on the x-axis values of HPLC as the reference method and on the y axis values of the known method as the comparable method (FIG. 10A), and values of the method subject of the invention are shown (FIG. 10B).

FIGS. 10C and 10D are Bland-Altman graphs that depict a comparison of indoxyl sulphate's assessment methodologies on a calibration set, for known method as the comparable method (FIG. 10C) and the method subject of the invention (FIG. 10D); where on the x-axis are mean values of HPLC as the reference method and comparable method (C(IS)_HPLC+C(IS)_Model)/2, and on y-axis are residuals between reference and comparable method C(IS)_HPLC−C(IS)_Model.

FIGS. 11A and 11B are graphs that depict a comparison of indoxyl sulphate's assessment methodologies on a validation set; where on the x-axis values of HPLC as the reference method and on the y axis values of the known method as the comparable method (FIG. 11A), and values of the method subject of the invention are shown (FIG. 11B).

FIGS. 11C and 11D are Bland-Altman graphs that depict a comparison of indoxyl sulphate's assessment methodologies on a validation set, for known method as the comparable method (FIG. 11C) and the method subject of the invention (FIG. 11D); where on the x-axis are mean values of HPLC as the reference method and comparable method (C(IS)_HPLC+C(IS)_Model)/2, and on y-axis are residuals between reference and comparable method C(IS)_HPLC−C(IS)_Model.

FIGS. 12A and 12B are graphs that depict a comparison of beta-2-microglobulin's assessment methodologies on a calibration set; where on the x-axis values of ELISA as the reference method and on the y axis values of the known method as the comparable method (FIG. 12A), and values of the method subject of the invention are shown (FIG. 12B).

FIGS. 12C and 12D are Bland-Altman graphs that depict a comparison of beta-2-microglobulin's assessment methodologies on a calibration set, for known method as the comparable method (FIG. 12C) and the method subject of the invention (FIG. 12D); where on the x-axis are mean values of ELISA as the reference method and comparable method (C(b2M)_ELISA+C(b2M)_Model)/2, and on y-axis are residuals between reference and comparable method C(b2M)_ELISA−C(b2M)_Model.

FIGS. 13A and 13B are graphs that depict a comparison of beta-2-microglobulin's assessment methodologies on a validation set; where on the x-axis values of ELISA as the reference method and on the y axis values of the known method as the comparable method (FIG. 13A), and values of the method subject of the invention are shown (13B).

FIGS. 13C and 13D are Bland-Altman graphs that depict a comparison of beta-2-microglobulin's assessment methodologies on a validation set, for known method as the comparable method (FIG. 13C) and the method subject of the invention (FIG. 13D); where on the x-axis are mean values of ELISA as the reference method and comparable method (C(b2M)_ELISA+C(b2M)_Model)/2, and on y-axis are residuals between reference and comparable method C(b2M)_ELISA−C(b2M)_Model.

FIGS. 14A and 14B are graphs that depict a comparison of 4-pyridoxic acid's assessment methodologies on a calibration set; where on the x-axis values of HPLC as the reference method and on the y axis values of the known method as the comparable method (FIG. 14A), and the method subject of the invention are shown (FIG. 14B).

FIGS. 14C and 14D are Bland-Altman graphs that depict a comparison of 4-pyridoxic acid's assessment methodologies on a calibration set for the known method as the comparable method (FIG. 14C) and the method subject of the invention (FIG. 14D); where on the x-axis are mean values of HPLC as the reference method and comparable method (C(4PA)_HPLC+C(4PA)_Model)/2, and on y-axis are residuals between reference and comparable method C(4PA)_HPLC−C(4PA)_Model.

FIGS. 15A and 15B are graphs that depict a comparison of 4-pyridoxic acid's assessment methodologies on a validation set; where on the x-axis values of HPLC as the reference method and on the y axis values of the known method as the comparable method (FIG. 15A), and values of the method subject of the invention are shown (FIG. 15B).

FIGS. 15C and 15D are Bland-Altman graphs that depict a comparison of 4-pyridoxic acid's assessment methodologies on a validation set for the known method as the comparable method (FIG. 15C) and the method subject of the invention (FIG. 15D); where on the x-axis are mean values of HPLC as the reference method and comparable method (C(4PA)_HPLC+C(4PA)_Model)/2, and on y-axis are residuals between reference and comparable method C(4PA)_HPLC−C(4PA)_Model.

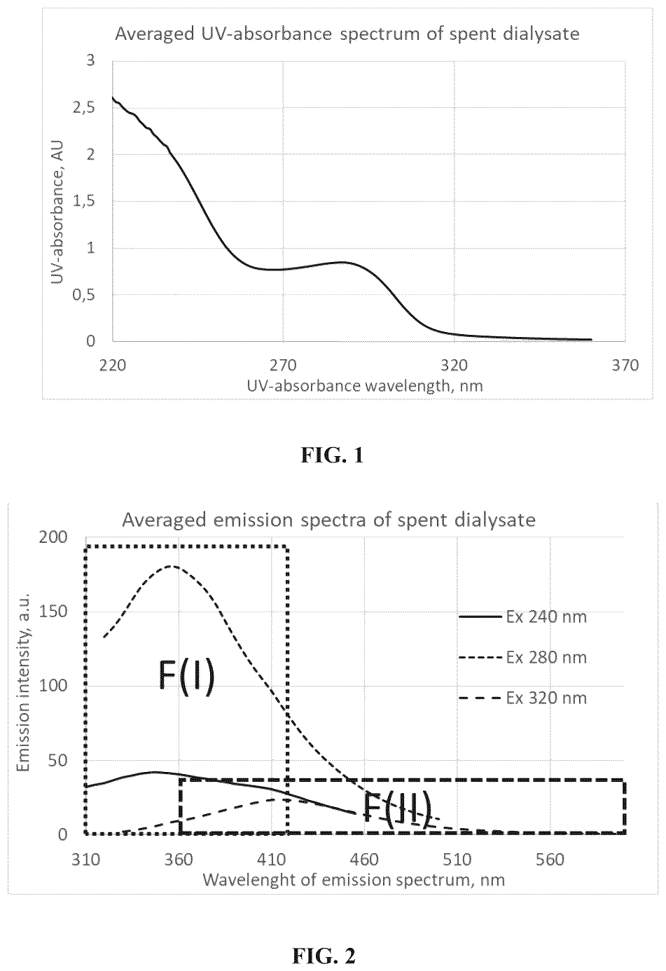

### DESCRIPTION OF THE INVENTION

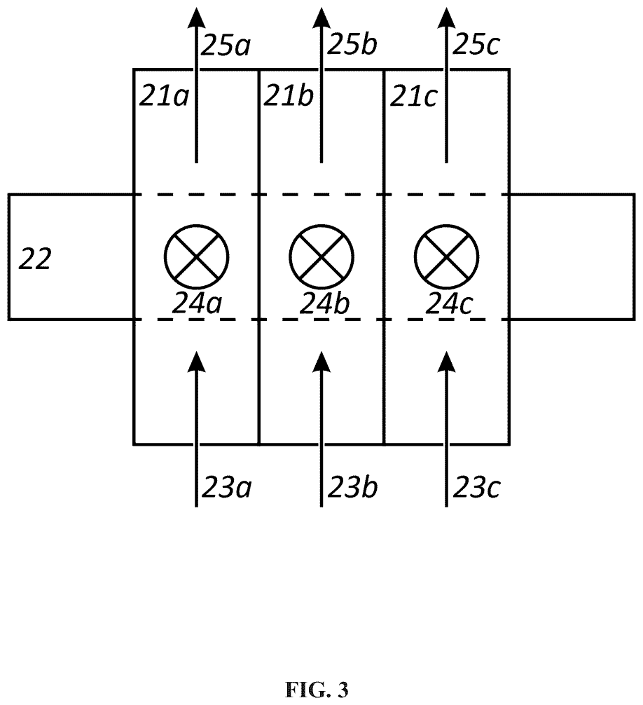

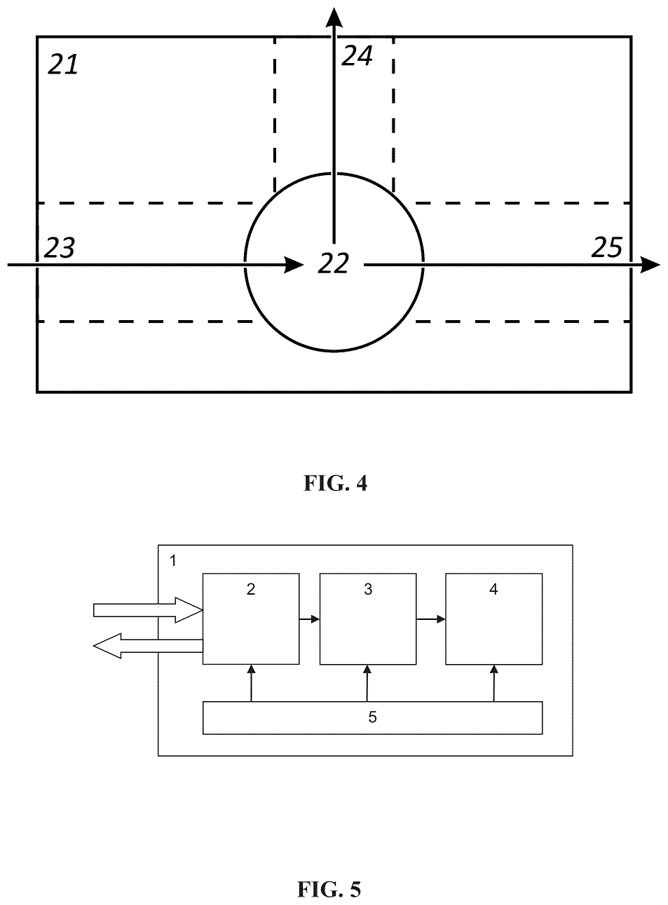

One of the possible embodiment examples of the device 1 according to the present invention comprises at least one optical measuring module 2, signal processing module 3, data communication and data representing module 4, and power supply unit and control device for supplying other modules with supply voltage and for controlling their work (see FIG. 5). Optical measuring module 2 is described in detail on FIGS. 3 & 4. Optical measuring module comprises of:
at least one modular optical measuring cell (in FIG. 3 shown as: 21a. 21b & 21c);
measuring cuvette 22 that passes through each measuring cell 21a, 21b, 21c for storing the biological fluid;
each modular measuring cell 21a, 21b, 21c comprises of a light source 23a, 23b, 23c for directing the light to the biological fluid of interest, light detector 24a, 24b, 24c for measuring the absorption of light in the biological fluid and light detector 25a, 25b, 25c for measuring fluorescent light that is emitted light from the biological fluid.

Signal processing module contains 3 data acquisition module and a computational device for signal processing.

Each modular optical measuring cell contains preferentially a light source with a maximum spectral bandwidth of 20 nm. Broadband light detector with optical filters, or narrowband light detector can be used as as a light detector. In one of the possible embodiments, the the light sources of optical measuring module operate in the ultraviolet radiation region (wavelength range of 230-380 nm). Wherein, the measuring cuvette of the device can be a flow-cuvette for receiving a flowing stream of the biological fluid or without flow through and a one open side.

For the calibration of the general design of the device, coefficients a0 . . . ai, b0 . . . bj, and c0 . . . ck are determined empirically for the Equations (1), (2) and (3) from the clinical trials, durin which reference concentrations are determined by laboratory reference methods. Coefficients that are determined are applicable for all of the devices that are based on the identical design. Each individual device can be calibrated with the reference solutions.

The advantage of the invention manifests in optically determining uremic solutes and uremic toxins concentration in biological fluids (including spent dialysate from hemodialysis) using multiparameter algorithm that does not require additional reagents and processing test solutions, whereas significantly improving the measurement accuracy compared to the closest solutions known from the state of the art. The input parameters of the method are areas of light absorption and fluorescence at wavelength regions, which are attributed to 1) peptide bonds of proteins; 2) specific amino acids in the composition of proteins; 3) absorbing solutes that have adsorbed to the surface of proteins; 4) fluorescent amino acids in the composition of proteins; 5) fluorescent substances sorbed on the surface of proteins; 6) fluorescent AGE-s. As an example of the this invention concentration determination of uremic toxins is presented for protein bound uremic toxins e.g. indoxyl sulphate, middle molecules, e.g. beta-2-microglobulin, and AGE-s, e.g 4-pyridoxic acid. in biological fluids, e.g. spent dialysate that is excreted from the dialysis machine.

The following dataset, which is given as an example, contains measurements results of spent dialysate samples of 22 end stage kidney disease patients that were collected during hemodialysis sessions. The study was approved by the Tallinn Medical Research Ethics Committee in Estonia (decision no. 2205, 27. Dec. 2017) and conducted in accordance with the Declaration of Helsinki. Patients were included into the study based on the following criteria: over 18 years old, on chronic hemodialysis, hemodialysis procedures via AV fistula or graft (catheters were not used) for 4 h thrice weekly, blood access capable to manage blood flow of at least 300 mL/min, absence of clinical signs of infection or other active acute clinical complications and an estimated life expectancy over 6 months. Clinical data of the participants were monitored for a total of 66 hemodialysis sessions Fresenius 5008 hemodialysis machines were used (Fresenius Medical Care, Bad Homburg v. d. Wife, Germany). Samples were collected from each patient during three midweek dialysis sessions, that used three different treatment settings: (1) hemodiafiltration (HDF) with standard settings previously prescribed for the patient in routine clinical care; (2) medium HDF with maximum dialyzer surface area and highest dialysate blood flow ratio (Qd/Qb); (3) high HDF with maximum dialysis settings in terms of dialyzer surface area, dialysate and blood flow, and the substitution volume.

Spent dialysate samples were taken from the dialysate outlet of the dialysis machine at 7, 60, 120 and 180 min after the start of the session and at the end of the session (240 min). In addition, the waste dialysate was collected into a large tank during the whole procedure to determine removed uremic toxins. After the end of the procedure, the dialysate collection tank was weighed, and one sample was taken from it after careful stirring. All dialysate samples were divided into two aliquots: the first set of samples were directly sent to a local clinical laboratory to conduct standard analysis (Synlab Eesti OÜ, Tallinn, Estonia); another sets of samples were analysed in the biochemistry laboratory of Department of Health Technologies in Tallinn University of Technology. Samples that were taken during self tests or errors of hemodialysis machine were omitted from the dataset.

Indoxyl sulphate was determined with the HPLC method that has been described in the publication of Arund et al. 2016. 4-Pyridoxic acid was determined with the HPLC method described in the publication of Kalle et al. 2016. Beeta-2-microglobulin was determined by the clinical laboratory Synlab Eesti OÜ using standard ‘sandwich’ type immunochemical system “Immulite2000 Beta-2 Microglobulin” (Siemens Healthineers AG, Erlangen, Germany).

UV-absorption spectra were recorded with the UV-3600 spectrophotometer (Shimadzu, Kyoto, Japan) in the wavelength range of 190-400 nm with the increment of 1 nm using a cuvette with optical path length of 10 mm. An untreated pure dialysis buffer was used as the reference solution, sampled from the outflow of the dialysis machine prior to switching on the blood flow. Fluorescence spectra were recorded with the spectrofluorometer RF-6000 (Shimadzu, Kyoto, Japan) using the excitation wavelength range of 200-400 nm with the increment of 10 nm and the emission wavelength range of 210-600 nm with the increment of 1 nm. The bandwidths of 5 nm were used in both monochromators and the used cuvette had an optical path length of 4 mm.

The following is a comparison of the effect of input parameters on the output of the method for selected uremic toxins, where the multiparametric method significantly improves both the concentration of reference points (coefficient of determination, R2) systematic deviation (BIAS) and the scattering of points (standard deviation, SE) in comparison with the reference method.

$$BIAS = \frac{{Σ}_{i = 1}^{N} {e}_{i}}{N} \qquad ( 4 )$$

$$SE = \sqrt{\frac{{Σ}_{i = 1}^{N} {( {e}_{i} - BIAS )}^{2}}{N - 1}} \qquad ( 5 )$$

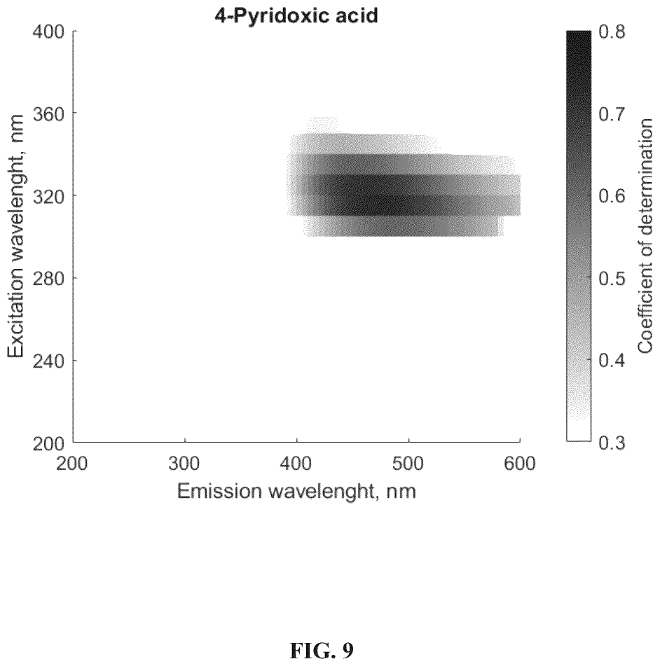

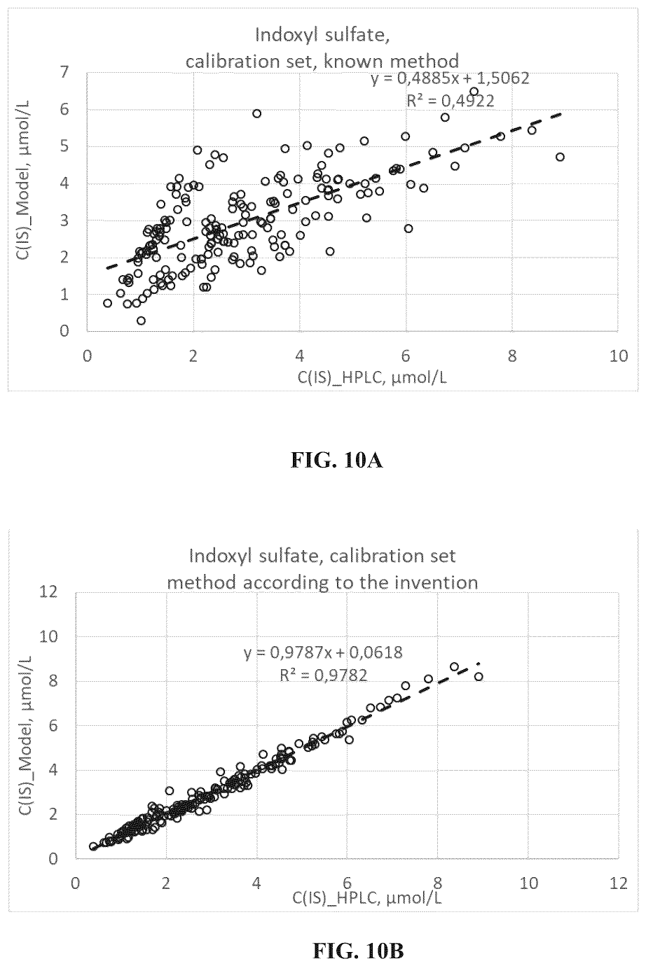

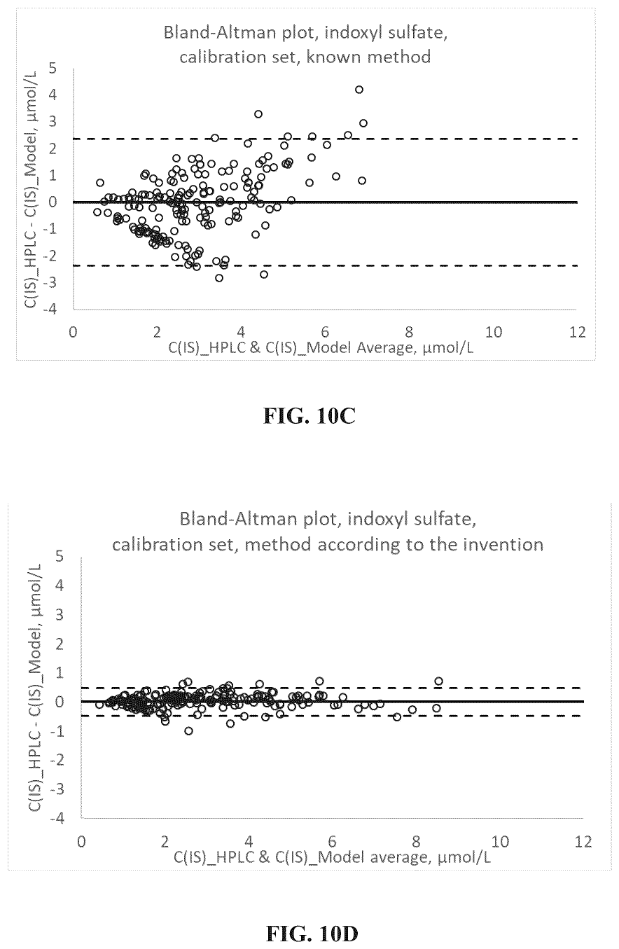

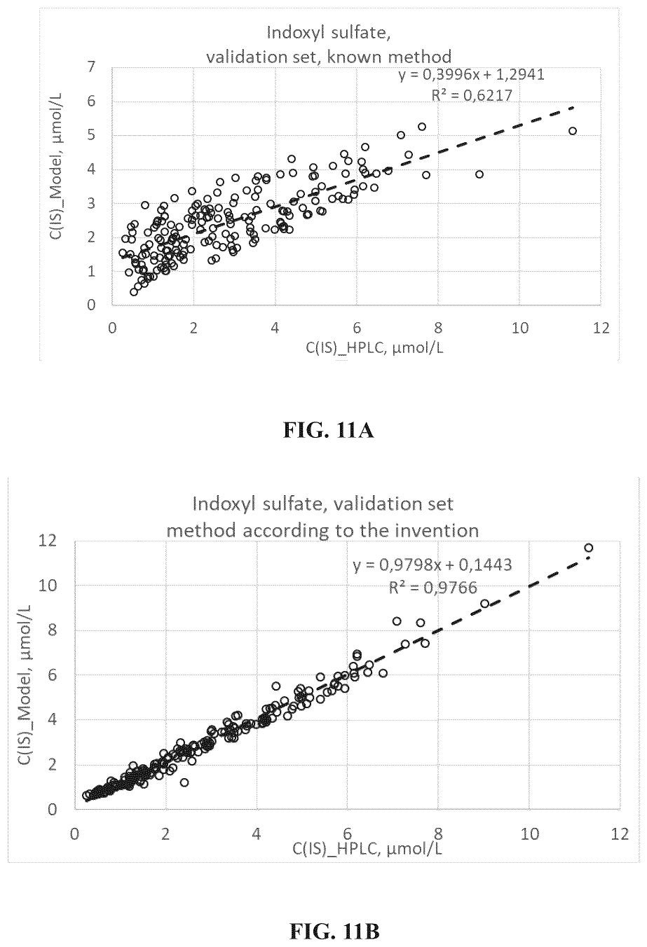

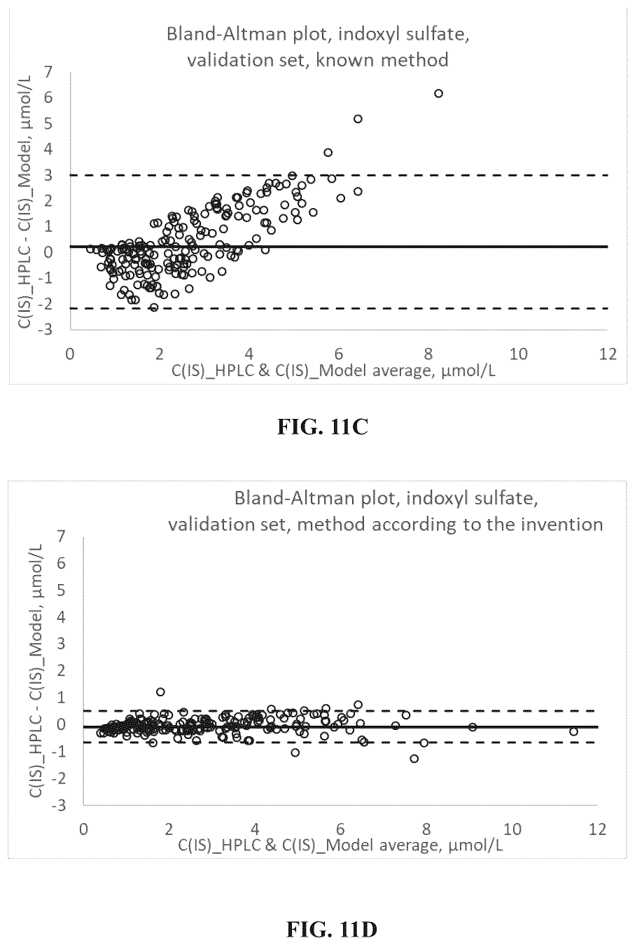

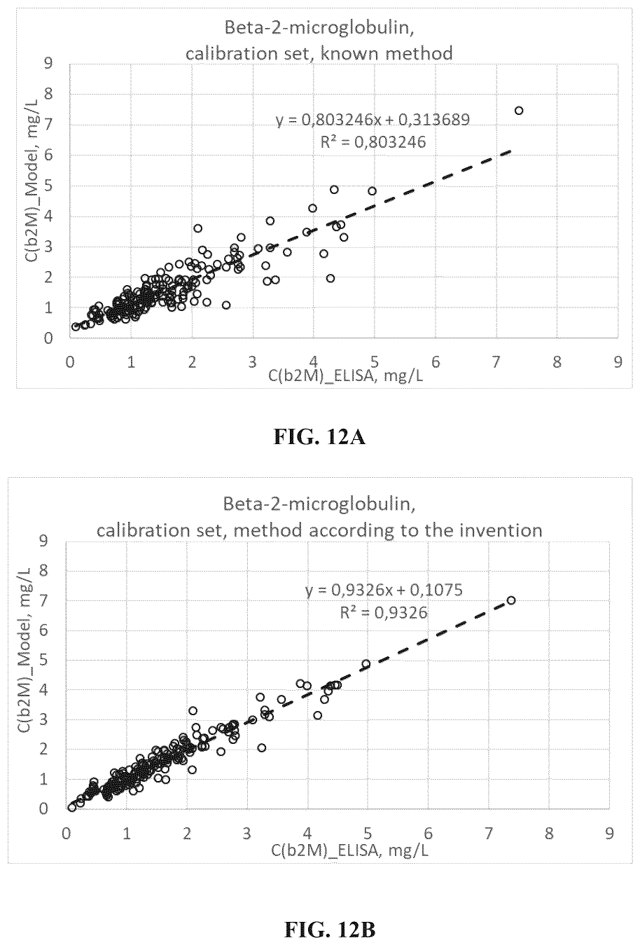

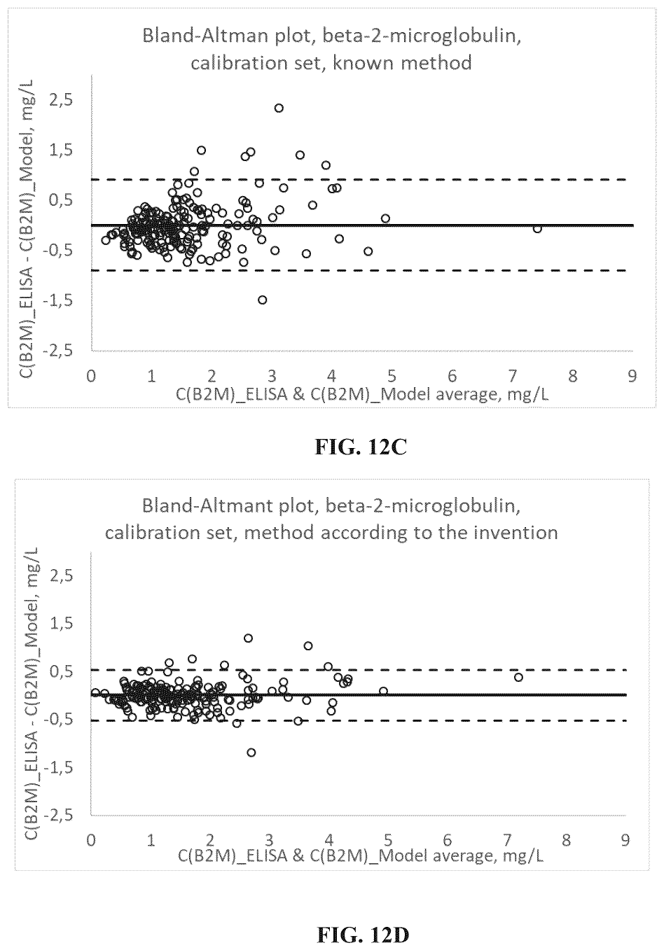

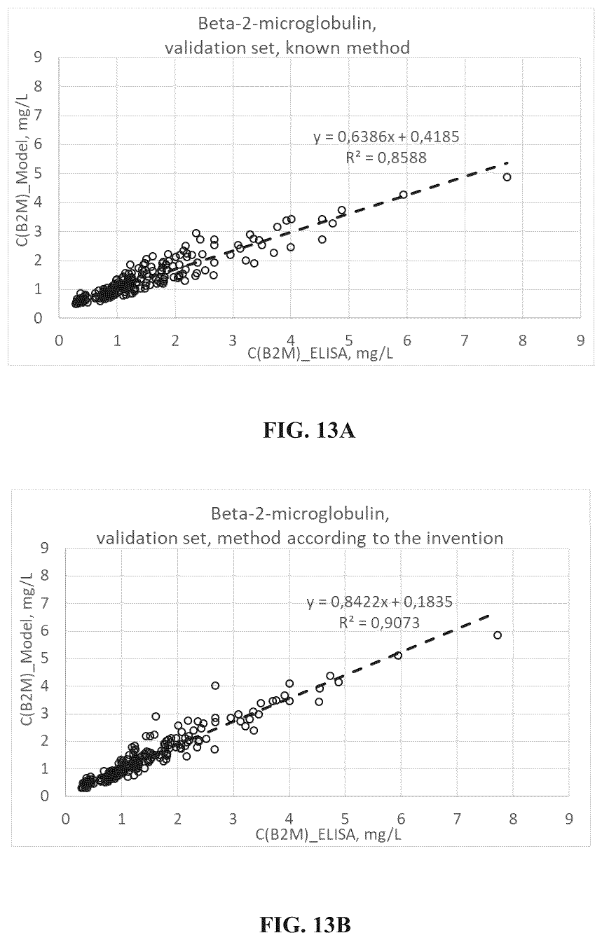

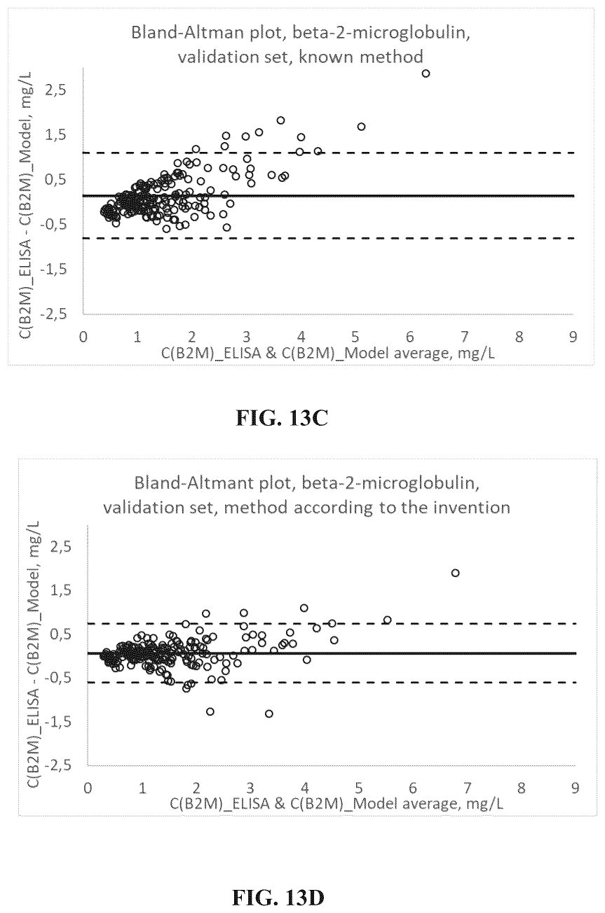

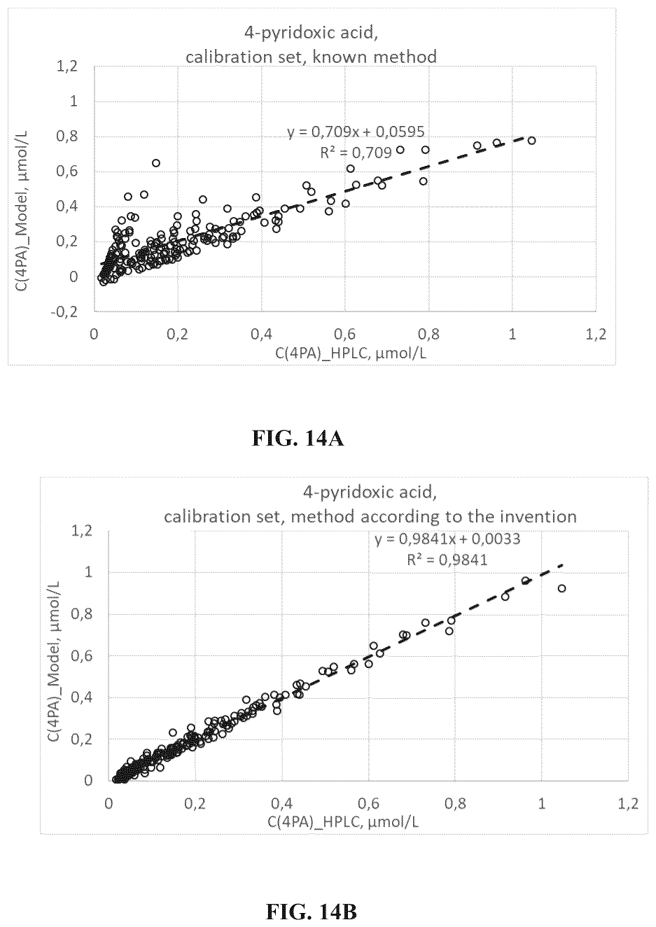

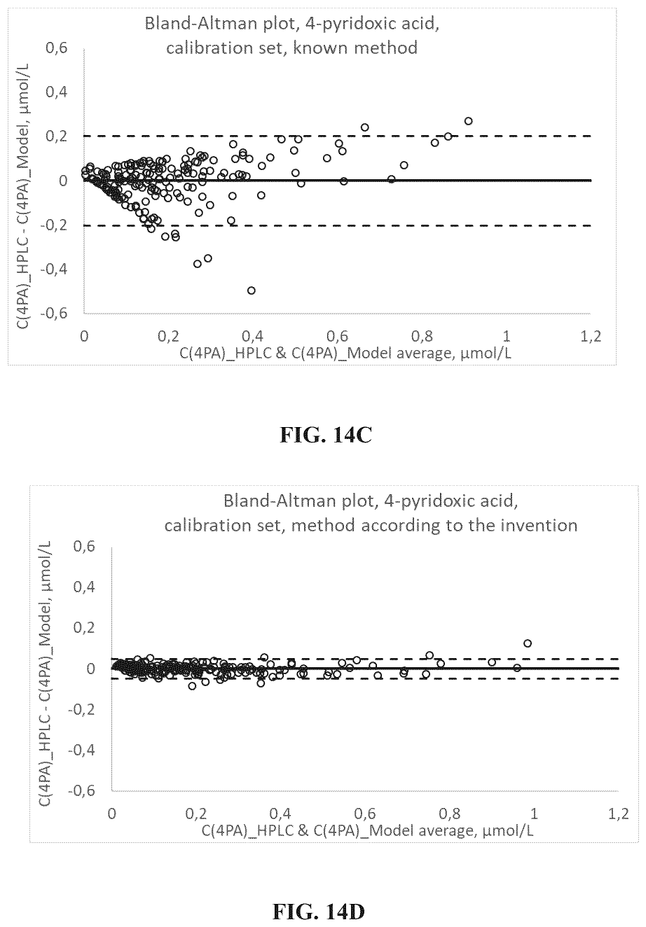

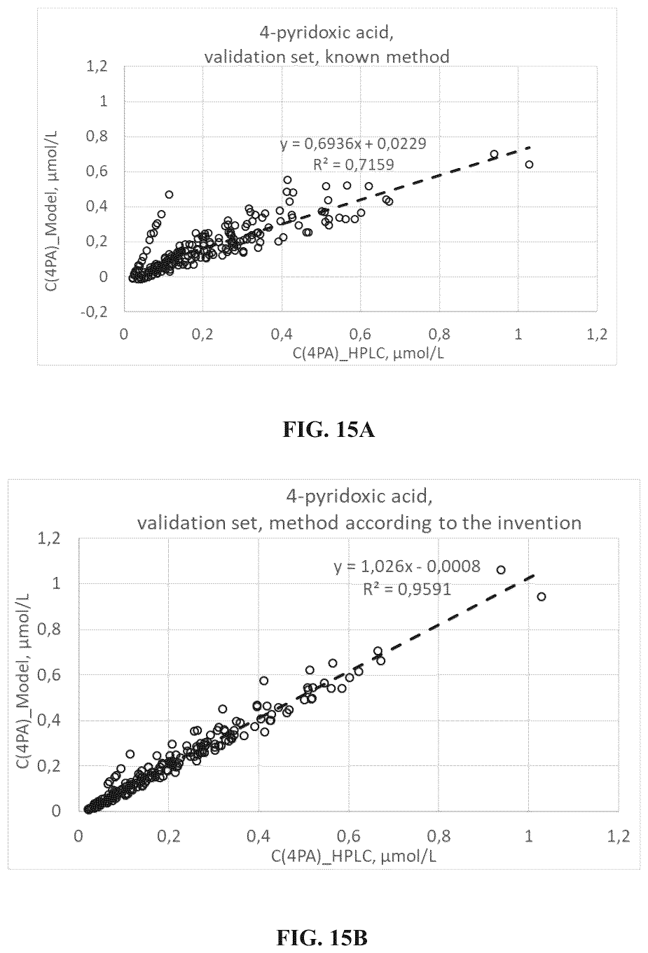

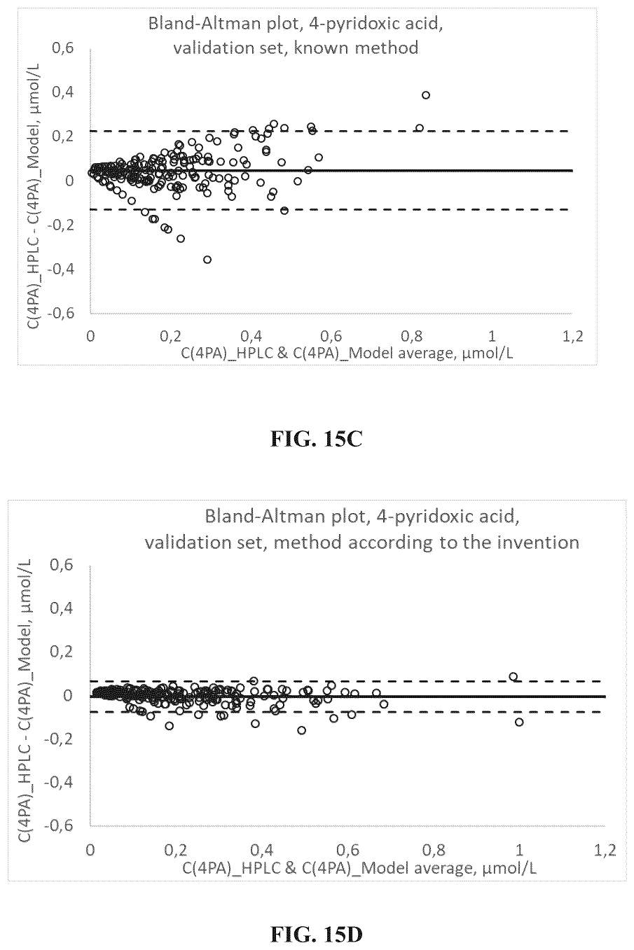

The data were used as a three different subsets: (i) all data together to analyse the effect of input parameters on a multiparametric model (Tables 1, 3, 5); and in the form of training and validation data, where (ii) the measurement data of 11 patients were in the calibration subset of the models; and measurement data of 11 patients in the model validation subset (Tables 2, 4, 6, FIGS. 10A to 15D).

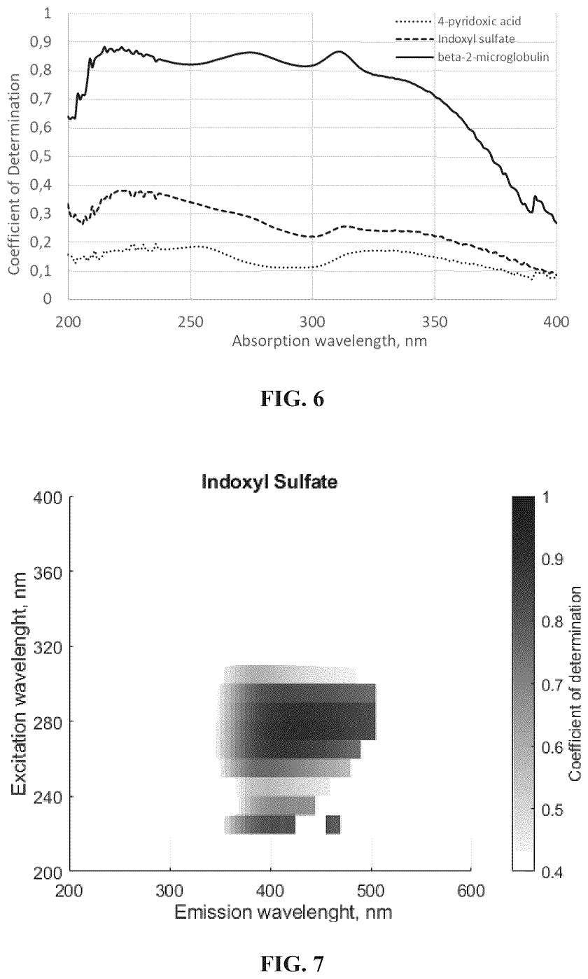

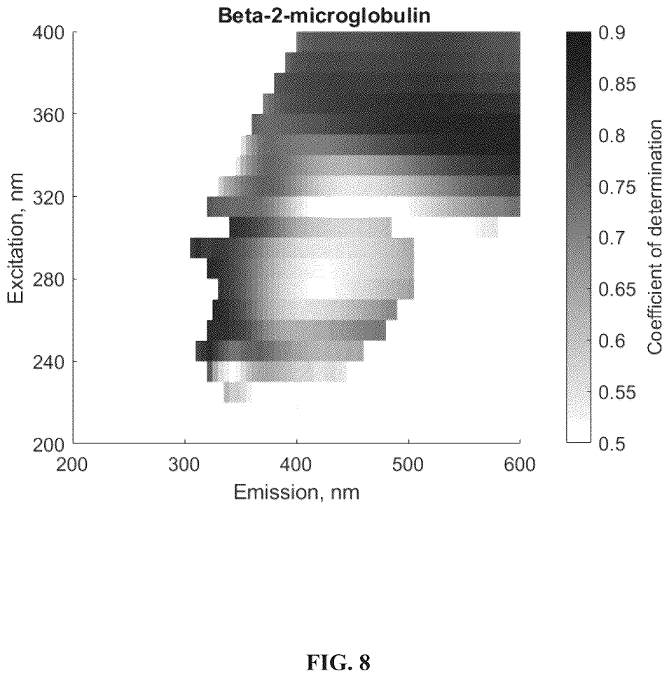

FIG. 6 depicts the strength of the linear relationship between uremic solutes concentration and absorption signal over absorption spectrum, in the form of coefficient of determination at different wavelengths.

Figures from 7 to 9 show the strength of the linear relationship between uremic solutes concentration and fluorescence signal; coefficient of determination R2 is given for different excitation and emission wavelengths.

On the first line with an asterisk, the results that were achieved with the previously known methods in a given data sample are shown, and on the last line the result that were obtained with a novel method:

Protein-bound uremic toxins—as example indoxyl sulphate, reference method liquid chromatography, HPLC (Tallinn University of Technology, Tallinn, Estonia)

The following model was generated with linear regression based on optical signals and known solution for assessing indoxyl sulphate:

C(IS)=−1,179+0,000161*F(Ex300Em355) (6)

Based on a novel method a new model was generated with multiparametric linear regression based on optical signals for assessing indoxyl sulphate:

C(IS)=−0,0312+0,000120*f(A240)*F(Ex240Em390)+ . . . 0,000119*f(A280)*F(Ex280Em425) (7)

TABLE 1
Influence of each parameter on the assessment of concentration of indoxyl
sulphate in the spent dialysate, number of datapoints n = 369.

| Input parameters, | Coefficient of | Accuracy (BIAS ± SE), | |
|---|---|---|---|
| absorption/fluorescence, nm | Determination | μM/L | |
| F(Ex300Em355)* | 0.522 | 0 ± 1.263 | Known method |
| F(Ex240Em390) | 0.067 | 0 ± 1.761 | |
| F(Ex280Em425) | 0.800 | 0 ± 0.817 | |
| F(Ex240Em390) + F(Ex280Em425) | 0.932 | 0 ± 0.471 | Invention |
| f(A240) * F(Ex240Em390) | 0.562 | 0 ± 1.206 | |
| f(A280) * F(Ex280Em425) | 0.900 | 0 ± 0.575 | |
| f(A240) * F(Ex240Em390) + f(A280) * | 0.977 | 0 ± 0.279 | Best model |
| F(Ex280Em425) | | | |

where F(Ex280Em425) is the florescence intensity of the measured fluid at excitation wavelength 280 nm and emission wavelength 425 nm, analogous for the following parameters; f(A280) is the correction function of primary inner-filter effect calculated at the absorption wavelength 280 nm (Wang et al. 2017). The result given in the first row in Table 1 is calculated with known method based on the patent EP 2 585 830 B1.

TABLE 2
Influence of each parameter on the assessment of
concentration of beta-2-microglobulin in the spent
dialysate, number of datapoints n = 375.

| | Calibration set | | Validation set | |
|---|---|---|---|---|
| | Known | | Known | |
| | method* | New method | method* | Uus meetod |
| Data points, n | 183 | 183 | 192 | 192 |
| R² | 0.492 | 0.978 | 0.619 | 0.977 |
| BIAS | −0.003 | 0.001 | 0.207 | −0.087 |
| SE | 1.206 | 0.250 | 1.203 | 0.298 |

Known method is based on the patent EP 2 585 830 B1. Results shown in Table 2 show that now multicomponent optical method improves the assessment accuracy more than 4 times (for the calibration set 1,206/0,250=4,82; for the validation set 1,203/0,298=4,03), and 2 times smaller systematic error (calibration set BIAS 0,207/0,087=2,38), compared to the known method.

Middle sized uremic toxins—as example beta-2-microglobulin, reference method ELISA (SYNLAB Eesti AS, Tallinn, Estonia)

The following model was generated with linear regression based on optical signals and known solution for assessing beta-2-microglobulin:

C(b2M)=0,160+0,000701*F(Ex370Em456) (8)

Based on a novel method a new model was generated with multiparametric linear regression based on optical signals for assessing beta-2-microglobulin:

C(b2M)=−0,372+1,125*A280+0,00000807*f(A280)*F(Ex280Em325)+0,00148*F(Ex350Em555) (9)

TABLE 3
Influence of each parameter on the assessment of concentration of
beta-2-microglobulin in the spent dialysate, number of datapoints n = 371.

| Input parameters, | Coefficient of | Accuracy (BIAS ± SE), | |
|---|---|---|---|
| absorption/fluorescence, nm | Determination | μM/L | |
| F(Ex370Em455)* | 0.809 | 0 ± 0.465 | Known |
| | | | method |
| A280 | 0.8535 | 0 ± 0.408 | |
| f(A280) * F(Ex280Em325) | 0.838 | 0 ± 0.428 | |
| F(Ex350Em555) | 0.8583 | 0 ± 0.401 | |
| A280 + f(A280) * F(Ex280Em325) | 0.882 | 0 ± 0.366 | |
| A280 + F(Ex350Em555) | 0.904 | 0 ± 0.329 | |
| f(A280) * F(Ex280Em325) + | 0.909 | 0 ± 0.322 | Invention |
| F(Ex350Em555) | | | |
| A280 + f(A280) * F(Ex280Em325) + | 0.931 | 0 ± 0.272 | Best Model |
| F(Ex350Em555) | | | |

where F(Ex370Em455) is the florescence intensity of the measured fluid at excitation wavelength 370 nm and emission wavelength 455 nm, analogous for the following parameters; A280 is the absorbance of the measured fluid at the wavelength of 280 nm; f(A280) is the correction function of primary inner-filter effect calculated at the absorption wavelength 280 nm (Wang et al. 2017). The result given in the first row in Table 3 is calculated with known method based on the patent EP 2 585 830 B1. New method provides 1,7 times (0,465/0,272=1,71) better accuracy compared to the known method which can be seen on Table 3.

TABLE 4
Comparison of known and new method to assess the concentration
of beta-2-microglobulin in the spent dialysate.

| | |
|---|---|
| Calibration set | Validation set |

| | Known | New | Known | New |
|---|---|---|---|---|
| | method | method | method | method |
| Data points, n | 183 | 183 | 192 | 192 |
| R² | 0.803 | 0.933 | 0.859 | 0.907 |
| BIAS | 0.000 | 0.000 | 0.141 | 0.061 |
| SE | 0.462 | 0.271 | 0.411 | 0.333 |

Known method is based on the patent EP 2 585 830 B 1. Results shown in Table 4 show that now multicomponent optical method improves the assessment accuracy more than 1,2 times (for the calibration set 0,462/0,271=1,70; for the validation set 0,411/0,333=1,23), and 2 times smaller systematic error (calibration set BIAS 0,141/0,061=2,31), compared to the known method.

Glycation End-Products (AGE products), as example 4-pyridoxic acid, reference method liquid chromatography, HPLC (Tallinn University of Technology, Tallinn, Estonia)

The following model was generated with linear regression based on optical signals and known solution for assessing 4-pyridoxic acid:

C(4PA)=−0,0323+0,0000226*F(Ex320Em440) (10)

Based on a novel method a new model was generated with multiparametric linear regression based on optical signals for assessing 4-pyridoxic acid:

C(4PA)=0,00160+−0,0000426*F(Ex320Em390)+0,0000594*F(Ex320Em440) (11)

TABLE 5
Influence of each parameter on the assessment of concentration of
4-pyridoxic acid in the spent dialysate, number of datapoints n = 371.

| Input parameters, | Coefficient of | Accuracy (BIAS ± SE), | |
|---|---|---|---|
| absorption/fluorescence, nm | Determination | μM/L | |
| F(Ex320Em440) | 0.696 | 0 ± 0.100 | Known method |
| F(Ex320Em390) | 0.345 | 0 ± 0.146 | |
| F(Ex320Em390) + | 0.972 | 0 ± 0.030 | Invention |
| F(Ex320Em440) | | | |

where F(Ex320Em390) is the florescence intensity of the measured fluid at excitation wavelength 320 nm and emission wavelength 390 nm, analogous for the following parameters. The result given in the first row in Table 5 is calculated with known method based on the publication Kalle et al. 2016.

TABLE 6
Comparison of known and new method to assess the concentration
of 4-pyridoxic acid in the spent dialysate.

| | Calibration set | | Validation set | |
|---|---|---|---|---|
| | Known | New | Known | New |
| | method | method | method | method |
| Data points, n | 182 | 182 | 189 | 189 |
| R² | 0.709 | 0.984 | 0.716 | 0.959 |
| BIAS | 0.0000 | 0.0000 | 0.0455 | −0.0050 |
| SE | 0.1039 | 0.0243 | 0.0906 | 0.0344 |

Known method is based on the publication of Kalle et al. 2016. Results shown in Table 6 show that now multicomponent optical method improves the assessment accuracy more than 2,5 times (for the calibration set 0,1039/0,0243=4,28; for the validation set 0,0906/0,0344=2,63), and 9 times smaller systematic error (calibration set 0,0455/0,005=9,10), compared to the known method.

Alternative known methods (such as HPLC, ELISA), that are commonly used in laboratories to assess the concentrations of the solutes mentioned hereby, are time-consuming, does not provide real-time measurement, need additional reagents and consumables. Known optical methods are not accurate enough for practical use. Novel multiparametric optical method enables to assess the uremic solutes in the fluids directly, without any manipulation, without additional reagents, in real time.

This invention provides a significant improvement for the assessment of the concentration of protein-bound and middle-sized uremic solutes and uremic toxins based on utilization of multicomponent input signals for novel algorithms.

The measurement is done with one cuvette in the device, device is preferably modular, where each module may consist of light sources with different parameters (such as light emitting diodes) and measurement elements (such as photomultipliers, phototransistors, and photodiodes), each module of the device is capable of measuring the signal of absorbance and fluorescence of the biological fluid simultaneously. The light sources of the device emits the light in the wavelength region of 190 nm and 400 nm and the measuring elements register the light in the region of 190 up to 800 nm.

### REFERENCES

A method and device for determining content of the middle and protein bound uremic toxins in a biological fluid https://patents.google.com/patent/EP2585830B1/

Device for determining waste products such as indoxyl sulphates in dialysis https://patents.google.com/patent/EP2746771B1/

Apparatus and method for the quantitative determination of urea, creatinine and uric acid in low molecular weight water-soluble substances https://patents.google.com/patent/EE05674B1/
Arund, J., Luman, M., Uhlin, F., Tanner, R., Fridolin, I., “Is Fluorescence Valid to Monitor Removal of Protein Bound Uremic Solutes in Dialysis?”,—PLOS ONE, 2016, 11, (5), e0156541, doi: 10.1371/journal.pone.0156541
Kalle et al. 2016. “4-pyridoxic Acid in the Spent Dialysate: Contribution to Fluorescence and Optical Monitoring”. PLoS ONE 11(9): e0162346
Lim et al. 2017. “Precise Quantitative Assessment of the Clinical Performances of Two High-Flux Polysulfone Hemodialyzers in Hemodialysis: Validation of a Blood-Based Simple Kinetic Model Versus Direct Dialysis Quantification“. Artificial Organs 2018 42(5): E55-E66.
Wang et al. 2017.” A review on the methods for correcting the fluorescence inner-filter effect of fluorescence spectrum”. Applied Spectroscopy Reviews, 52:10, 883-908
Watson et al. 1980. “Total body volumes for adult males and females estimated from simple anthropometric measurements”. Am J Clin Nurt 1980; 33; 27-39

What is claimed is:

1. A multiparametric optical method for assessing concentration of uremic solutes, including uremic toxins in biological solutions wherein the biological solution is transferred into measurement quvette, biological solution is illuminated with optical signal in the quvette, transmitted light from the biological solution is registered with detector, and concentration of uremic solutes in the biological solution is assessed with program executed in the computing device characterized in that optical signal is registered at least at two wavelength ranges at absorbance and/or fluorescence spectral optimums of the uremic solutes;
light absorbance and fluorescence are assessed from the light transmitted from the biological fluid at suitable wavelengths; and
concentrations of protein-bound and middle-sized uremic solutes, and glycation end-products, including indoxyl sulfate sulphate, beta-2-microglobulin, 4-pyridoxic acid, are assessed with multiparametric algorithm according to equation
C(X)=a₀+a₁*A₁b₁*f(A₁)*F(F₁)+a₂*A₂+b₂*f(A₂)*F(F₂)+ . . . +a₃*A₃+b₃*f(A₃)*F(F₃)+ . . . +aₙ*Aₙ+bₙ*f(Aₙ)*F(Fₙ), wherein
C(X) is the concentration of uremic solute, such as indoxyl sulphate, beta-2-microglobulin, or 4-pyridoxic acid in μmol/L or mg/L,
aᵢ is empirically determined coefficients from the clinical trials;
Aᵢ is the absorption assessed from the biological solution at wavelength optimum area Aᵢ;
f(Aᵢ) is the correction function of the primary inner-filter effect at the absorbance wavelength range Aᵢ;
F(Fᵢ) is the fluorescence of the biological solution at the wavelength range Fᵢ; and
calculated concentrations are transferred to the communication module and/or display device.

2. The method according to claim 1, characterized in that:
light absorbance A₁ . . . Aₙ in the biofluids is determined at the wavelength λ range 230-300 nm that corresponds to the absorbance of chromophores (uric acid, hippuric acid); functional groups in the protein (tryptophan, tyrosine, phenylalanine); and sorbants bound to proteins (hippuric acid, p-cresylsulfate, indoxyl sulphate);
light fluorescence F₁ . . . Fₙ in the biofluids is determined at the wavelength range that corresponds to the fluorescent uremic solutes (indoxyl sulphate, indole-acetic acid, p-cresyl sulphate); fluorescent amino acids consisting in the protein structure (tryptophan, indoxyl sulphate, p-cresyl sulphate); and fluorescent sorbants bound to proteins (tryptophan, indoxyl sulphate, p-cresyl sulphate), wherein said excitation light wavelength λEx is in the range of 240-310 nm, emitting light wavelength λEm is in the range of 270-420 nm;
light fluorescence F₁ . . . Fₙ in the biofluids is determined at the wavelength range that corresponds to the fluorescence of glycation end-products (AGE) (including 4-pyridoxic acid, AGE pentosidine, AGE argpyrimidine, AGE vesperlysine, wherein said excitation light wavelength λEx is in the range of 310-390 nm, emitting light wavelength λEm is in the range of 360-600 nm; and
empirically determined coefficients from the clinical trials for the parameters of A₁ . . . Aₙ ja F₁ . . . Fₙ.

3. A device for assessing the constituent and concentration of middle-sized and protein-bound uremic solutes in the biological fluid that consists of:
cuvette for holding or flow-through of the biological fluid;
at least of one optical measurement cell;
wherein each measurement cell consists of a light source for illuminating the biological fluid and at least two light detectors to detect emitting light from the biological fluid; and
a device for signal processing which consists of module for data acquisition, computing device for data processing, and communication device for data transfer to the display device and/or external device/network, characterized in that the device is adapted to execute of the method described in claim 1.

4. The device according to claim 3, characterized in that at least two light sources are emitting light in the wavelength range of 230-380 nm.

5. The device according to claim 3, characterized in that at least two light detectors are detecting light in the wavelength range of 230-600 nm.

6. The device according to claim 3, characterized in that the device is functioning on-line preferably.

7. The device according to claim 3, characterized in that one light source emits light in the wavelength range of 230-260 nm, one light detector detects the light in the range of 360-420 nm, second light source emits light in the wavelength range of 260-300 nm; and
second light detector detects the light in the range of 390-460 nm which is suitable for assessing the protein-bound uremic toxins, including indoxyl sulfate.

8. The device according to claim 3, characterized in that one light source emits light in the wavelength range of 230-260 nm, one light detector detects the light in the range of 230-260 nm, second light detector detects light in the wavelength range of 360-420 nm, second light source emits light the light in the range of 260-300 nm, third light detector detects the light in the range of 260-300 nm, fourth light detector detects light in the wavelength range of 390-460 nm, which is suitable for assessing the protein-bound uremic toxins, including indoxyl sulfate.

9. The device Dev′rec according to claim 3, characterized in that one light source emits light in the wavelength range of 260-290 nm, one light detector detects the light in the range of 260-290 nm, second light detector detects light in the wavelength range of 290-360 nm, second light source emits light the light in the range of 320-380 nm, third light detector detects the light in the range of 470-600 nm, which is suitable for assessing the middle sized uremic toxins, including beta-2-microglobulin.

10. The device according to claim 3, characterized in that one light source emits light in the wavelength range of 310-330 nm, one light detector detects the light in the range of 310-330 nm, second light detector detects light in the wavelength range of 360-420 nm, third light detector detects the light in the range of 410-600 nm, which is suitable for assessing the glycation end products (AGEs), including 4-pyridoxic acid.

11. The device according to claim 3, characterized in that the device is configured to provide the concentrations of protein-bound and middle sized uremic solutes and glycation end-products, including indoxyl sulfate, beta-2-microglobulin, 4-pyridoxic acid in the biological fluids in real-time, with time delay not more than 1 min, preferably not more than 1 s.

12. The device according to claim 3, characterized in that the computing device is configured to use the algorithms to calculate the quantity of a uremic solute that has passed through of the measurement cuvette according to the equation
TRS(x)=Σᵣ^{t}[C(Xₜ)*Q], wherein
TRS(x) is the quantity μmol, mmol, mg or g of a solute x that has passed the cuvette during the time 0 until time t;
t is the duration of the assessment; C(Xₜ) is the concentration of the uremic solute at the moment of t calculated by the the computing device; and
Q is the flow speed of the biological fluid flowing through the measurement cuvette.

13. The device according to claim 3, characterized in that the computing device is configured to use the algorithms to calculate the Time Averaged Concentration (TAC) of a uremic solute in the biological fluid of the spent dialysate that passes the measurement cuvette during the kidney replacement therapy procedure according to the equation (him et al. 2017)
TAC(x)=TRS(x)/(Kt/V*V_{Watson}), wherein
TAC(x) is the Time Averaged Concentration of a uremic solute x in μmol/L, mmol/L or mg/L;
TRS(x) is the quantity μmol, mmol, mg or g of a solute x that has been oved during the kidney replacement therapy;
Kt/V is the value to quantify the kidney replacement therapy with the simplified kinetical model; and
V_{Watson} is the volume of water according to Watsoni equation Watson et al. 1980).

14. The device for assessing the constituent and concentration of middle-sized and protein-bound uremic solutes in the biological fluid that consists of:
cuvette for holding or flow-through of the biological fluid;
at least of one optical measurement cell;
wherein each measurement cell consists of a light source for illuminating the biological fluid and at least two light detectors to detect emitting light from the biological fluid; and
a device for signal processing which consists of module for data acquisition, computing device for data processing, and communication device for data transfer to the display device and/or external device/network, characterized in that the device is adapted to execute of the method described in claim 2.
